(19) United States
(12) Patent Application Publication
(10) Pub. No.: US 2016/0339167 A1
(43) Pub. Date: Nov. 24, 2016

(54) INFUSION PUMP DRUG DELIVERY PROFILES, SYSTEMS, AND METHODS

(71) Applicant: SMITHS MEDICAL ASD,INC., Plymouth, MN (US)

(72) Inventors: Rick LEDFORD, Plymouth, MN (US); Larry ZALESKY, Plymouth, MN (US); Michael BLOMQUIST, Plymouth, MN (US); William ROSSI, Plymouth, MN (US); Jim DROST, Plymouth, MN (US); David A. SCANLON, Plymouth, MN (US); Kurt D. BERGQUIST, Plymouth, MN (US); Scott STRAW, Plymouth, MN (US)

(73) Assignee: Smiths Medical ASD, Inc., Plymouth, MN (US)

(21) Appl. No.: 15/114,500

(22) PCT Filed: Mar. 3, 2015
(86) PCT No.: PCT/US2015/018462

§ 371 Date: Jul. 27, 2016

Related U.S. Application Data
(60) Provisional application No. 61/949,667, filed on Mar. 7, 2014.

(51) Int. Cl. A61M 5/145; G06F 19/00

(52) U.S. Cl. CPC A61M 5/1452; G06F 19/3468; A61M 2005/14208

(57) ABSTRACT

Embodiments relate generally to drug delivery infusion profiles that can be transferred and executed on infusion pumps without the need for loading new firmware. Particularly, embodiments relate to integrating and/or editing delivery profiles for execution infusion systems. Such systems can include target-controlled infusion systems. Advantages of such embodiments include enabling healthcare providers and medical device companies to respond quickly to changes in infusion pump technology, drug development, and pharmacokinetics by providing the ability to create, edit, integrate, and store delivery profiles to accommodate these changes.

### RELATED APPLICATION

This application claims the benefit of U.S. Provisional Patent Application No. 61/949,667 filed Mar. 7, 2014, which is incorporated herein by reference in its entirety.

### TECHNICAL FIELD

Embodiments relate generally to drug delivery infusion profiles for infusion pumps and, more particularly, to drug delivery profiles that can be transferred, edited, and executed on infusion pump systems without a need for loading new firmware.

### BACKGROUND

Infusion pumps are extremely useful medical devices for managing the delivery and dispensation of therapeutic medications. Infusion pumps provide significant advantages over manual administration by accurately delivering medications over an extended period of time. Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large volume, patient-controlled analgesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an intraoperative site, epidural space or subarachnoid space.

Typically, infusion pumps are locally controlled via the programming of the individual infusion pump. For example, a physician can configure an infusion pump to execute a delivery profile that corresponds to a patient's treatment needs, or a patient can configure an infusion pump according to their individual requirements within pre-defined limits without the involvement of a physician. Generally, an infusion pump is programmed or configured according to certain physiological, pharmacokinetic, and operational parameters or limits that are often predefined. In recent years, infusion pumps have become increasingly sophisticated and may include such features as dose error reduction software, which enable infusion pumps to perform functions that assist healthcare providers with programming and calculating dose and delivery rates in an effort to reduce medication errors and potentially consequential harm to the patient. Infusion pumps can also be programmed or configured to access databases (often referred to as “drug libraries”) containing information relating to medications that can be used with that pump, as well as information corresponding to dosing guidelines, drug concentrations, dose limits, and clinical advisories. Such features can include computer- and/or server-based software that creates, configures or otherwise provides medication safety software settings. These features may generally enable healthcare providers to select medications from pre-loaded lists, which can be tailored to each healthcare facility and patient care area. Additionally, healthcare facilities can integrate infusion pumps with electronic medical records, computerized order entry systems, and medication recognition systems, such as, e.g., barcode scanning systems, to further enhance safety and efficacy. Healthcare facilities can also choose to generally and/or specifically implement dosing and delivery limitations, commonly called hard and soft limits, on preselected drugs.

As infusate therapies advance, there is a correspondingly increased need for infusion pumps that accommodate the evolving needs of patients, healthcare providers, and healthcare facilities. Improved infusion pump systems and methods should have the capability to integrate existing information concerning drug protocols and delivery profiles with new drugs and delivery profiles in a manner that is convenient and not dependent on a specific device or technology. Additionally, improved infusion pump systems and methods should have the capability to transition between different infusion pump protocols and mimic or emulate the protocols and regimes employed by various infusion pump manufacturers.

It would therefore be advantageous to provide an ability to access and download a database of information with which to execute delivery protocols for infusion pumps without the need for loading new firmware. It would also be advantageous to have an ability to readily transfer predefined infusion profiles, and edit and/or integrate the profiles, in order to adapt to changes in technology and pharmacology.

### SUMMARY

Embodiments described or otherwise contemplated herein substantially meet the aforementioned needs; for example, providing methods and systems for creating, integrating, editing, and storing infusion pump delivery profiles and profile segments. In an embodiment, an infusion pump is configured with subsystem software to download, edit, and execute infusion profiles or profile segments without a need for loading new firmware.

In an embodiment, predefined delivery profiles, such as target-controlled infusion (TCI) profiles and information relating delivery profiles can be collected and stored in a database or library. Embodiments of the database can include information relating to, for example, various drugs and pharmacokinetic parameters used to execute delivery protocols for use in an infusion pump context. Embodiments can include predefined delivery profiles, such as TCI profiles that can be transferred to an infusion pump, edited, and/or integrated and then executed on the infusion pump, without limitations as to the source of the delivery profiles or the manufacturer of the device typically used to execute the delivery protocols. In other embodiments, the delivery profiles can be segmentable, such that the user of an infusion pump can transfer entire profiles or segments of profiles to an infusion pump. One skilled in the art will readily understand that reference to TCI profiles throughout this disclosure is simply an exemplary embodiment used as an example, and is not intended to limit the scope, applicability, or configuration of the invention in any way. Rather, TCI is one example of the disclosure herein describing the adjustment of delivery profiles with respect to various parameters and/or behaviors of various infusates.

In a feature and advantage of embodiments, a user can integrate general or specific delivery profiles, or segments of delivery profiles, to create segmented delivery profiles, which can then be edited using the infusion pump. For example, each profile segment can be defined with a name, a set of delivery shape parameters, and/or user-defined parameters, which can be used as an underlying template for execution of delivery profiles or segments thereof, commonly supported by infusion pumps and associated systems. A user can alter a delivery profile by changing the coefficients of its underlying polynomial equation, or by adjusting the curve of the profile by interacting with a user interface (i.e., a touchscreen or keypad) on an infusion pump. In an embodiment, the delivery profiles created by a user can be stored on the infusion pump, or uploaded to a server for a database of other delivery profiles. Embodiments allow healthcare providers and medical device companies to respond quickly to changes in infusion pump technology, drug development, and pharmacokinetics by providing the ability to create, edit, integrate, and store delivery profiles to accommodate these changes.

The above summary is not necessarily intended to describe each illustrated embodiment or every implementation of the subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments of the subject matter in connection with the accompanying drawings, in which:

FIG. 1A is a perspective view of a syringe type infusion pump, according to an embodiment.

FIG. 1B is a front view of an ambulatory type infusion pump, according to an embodiment.

FIG. 2 is a diagram of an infusion pump system, according to an embodiment.

FIGS. 3A-3C are graphical representations of infusion pump delivery profiles and profile segments, according to an embodiment.

FIG. 4A graphically represents a collection of infusion pump profiles, according to an embodiment.

FIG. 4B represents various methods of transferring infusion pump profiles and related information, according to an embodiment.

FIG. 5 is a block diagram of a communications network, according to an embodiment.

FIG. 6 is a flowchart of a process for downloading and executing infusion pump delivery profiles, according to an embodiment.

FIG. 7 is a flowchart of a process for downloading, editing, and storing infusion pump delivery profiles, according to an embodiment.

FIG. 8 is a flowchart for operating a TCI system, according to an embodiment.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION

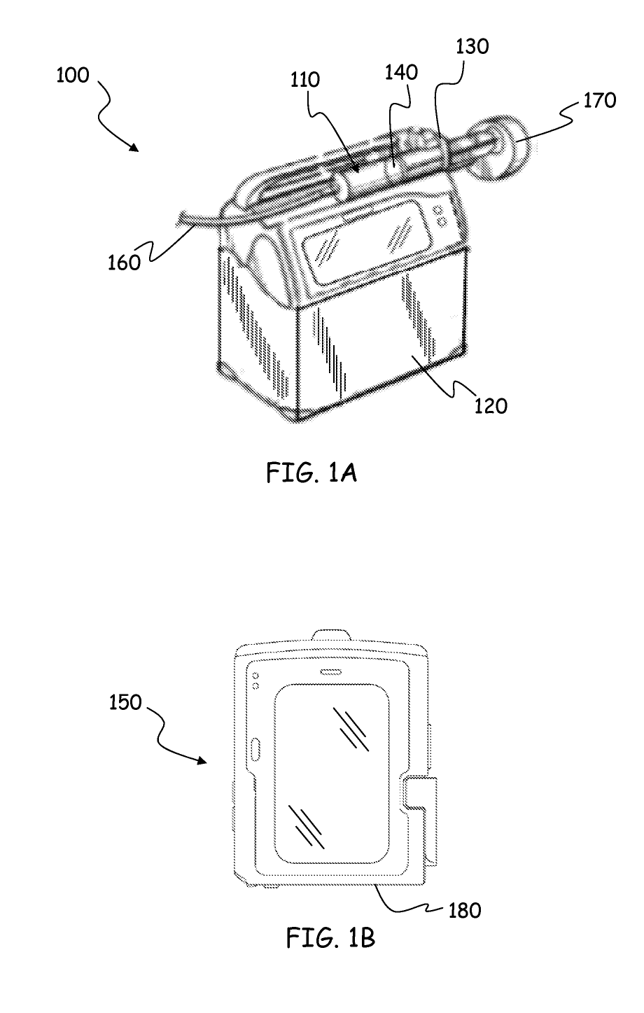

FIGS. 1A and 1B show examples of infusion pumps 100 and 150, respectively (also referred to more generally in this disclosure by numeral 100), which can be used to implement embodiments of the systems and methods discussed herein. In general, infusion pump 100 is a syringe-type pump that can be used to deliver a wide range of drug therapies and treatments. Infusion pump 100 includes a pharmaceutical container or syringe 110, which is supported on and secured to housing 120 by clamp 130, respectively. In embodiments, syringe 110 can be separately supplied from pump 100. In other embodiments, syringe 110 is an integrated component of pump 100. Syringe 110 includes a plunger 140 that forces fluid outwardly from syringe 110 via infusion line 160 that is connected to a patient. A motor and lead screw arrangement internal to housing 120 of pump 100 cooperatively actuates a pusher or plunger driver mechanism 170, to move plunger 140. In embodiments, a sensor (not shown; which is typically internal to plunger driver mechanism 170) monitors force and/or plunger position in the syringe according to system specifications.

Infusion pump 150 shown in FIG. 1B is an example of an ambulatory infusion pump that can be used to deliver a wide range of drug therapies and treatments. Such ambulatory pumps can be comfortably worn by or otherwise removably coupled to a user for in-home ambulatory care by way of belts, straps, clips or other simple fastening means, and can also be alternatively provided in ambulatory pole-mounted arrangements within hospitals and other medical care facilities. Infusion pump 150 generally includes a peristaltic type infusion pump mechanism that controls the flow of medication from a reservoir (not shown in FIG. 1B) of fluid coupled to pump 150 through a conduit from the reservoir which matingly passes along bottom surface 180 of pump 150. The reservoir can comprise a cassette that is attached to the bottom of pump 150 at surface 180, or an IV bag or other fluid source that is similarly connected to pump 150 via an adapter plate (not shown) at surface 180. Specifically, pump 150 uses valves and an expulsor located on bottom surface 180 to selectively squeeze a tube of fluid (not shown) connected to the reservoir to effect the movement of the fluid supplied by the reservoir through the tube and to a patient in peristaltic pumping fashion. Infusion pumps 100 and 150 are two examples of infusion pumps that can be suitable for use with embodiments discussed herein, though other pumps and devices can be used in other embodiments of infusion systems utilizing subject matter hereof.

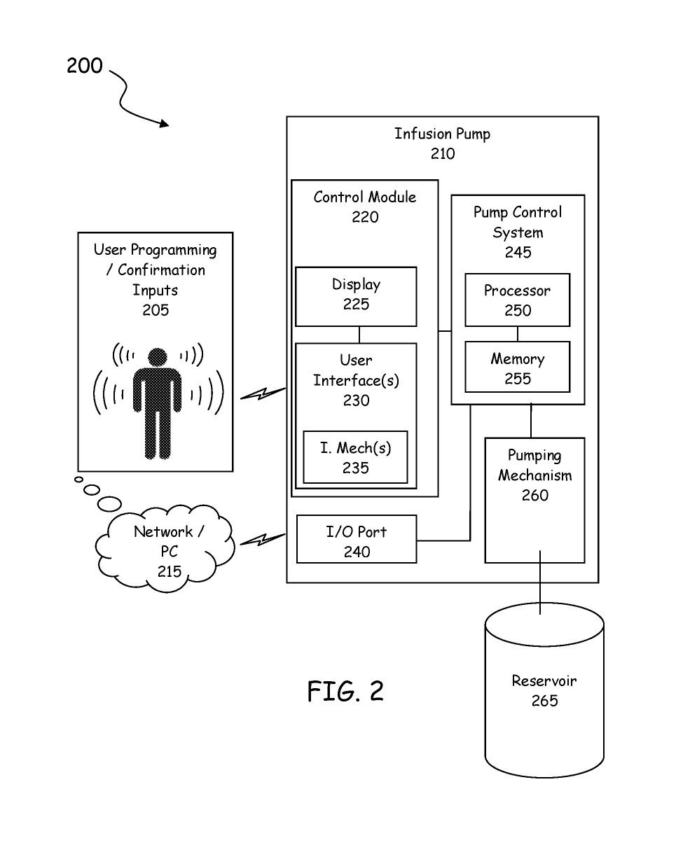

FIG. 2 is a schematic diagram of an infusion pump system 200. System 200 includes infusion pump 210 having pump control system 245 with processor 250 and memory 255 programmable with selected protocols, profiles, segments of profiles, and other settings for controlling operation of pumping mechanism 260 such as, for example, the aforementioned syringe and ambulatory or peristaltic type mechanisms. Infusion pump 200 can also include control module 220 (e.g., a user interface) for relaying commands to pump control system 245. Control module 220 includes at least one user interface 230 utilizing operator input technology including input mechanism(s) 235, which work with display screen 225. In some cases display 225 will be considered part of user interface(s) 230. User interface 230 generally allows a user to enter or select various parameters, including but not limited to names, drug information, limits, delivery shapes, information relating to hospital facilities, as well as various user-specific parameters (e.g., patient age and/or weight). Infusion pump 210 can include USB port or other appropriate input/output (I/O) interface port 240 for connecting infusion pump 210 to network or computer 215 having software designed to interface with infusion pump 210. Power to infusion pump 210 is accomplished via an AC power cord or an internally provided battery source. User inputs 205 to the system can be provided by programming from a user, such as a patient, pharmacist, scientist, drug program designer, medical engineer, nurse, physician, or other medical practitioner or healthcare provider. User inputs 205 may utilize direct interfacing (i.e., a keyboard or other touch-based inputs) or user inputs 205 may utilize indirect or “touchless” interfacing (i.e., gestures; voice commands; facial movements or expressions; finger, hand, head, body and arm movements; or other inputs that do not require physical contact). User inputs 205 are generally interfaced, communicated, sensed, and/or received by operator input mechanisms 235 of user interface 230. Operator input mechanisms 235 may include, for example, keyboards, touchscreens, cameras, or sensors of electric field, capacitance, or sound.

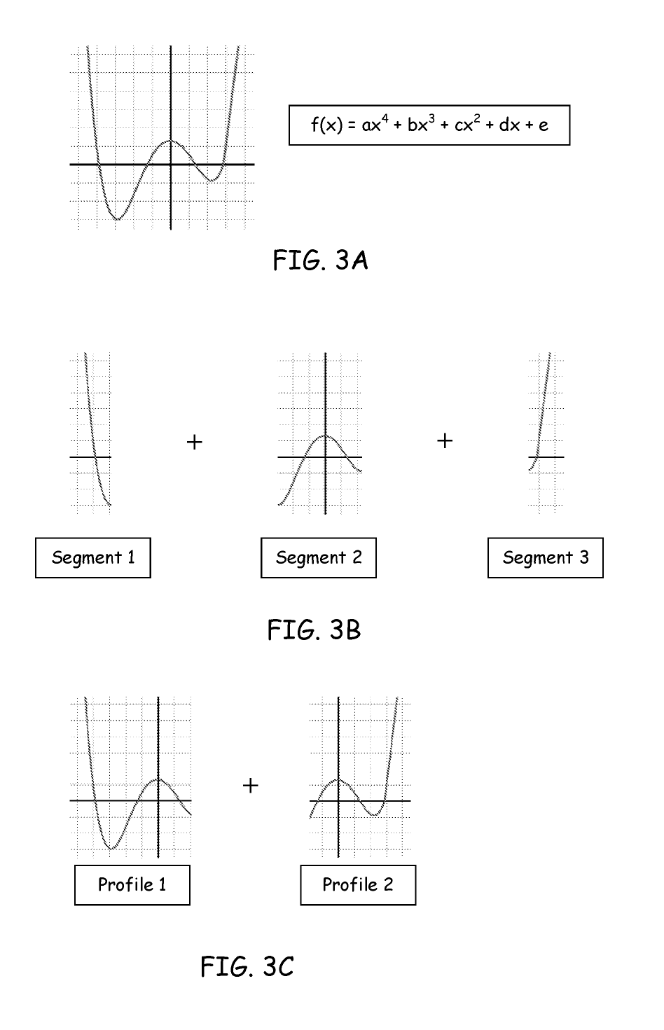

FIGS. 3A-3C include graphical representations of infusion pump delivery profiles and profile segments. In embodiments, a delivery profile generally comprises or defines the segments and segment interaction rules that are associated with a delivery method used by a drug protocol. Each segment has settings, and a drug protocol, or protocol, can be an overall group of settings that can be selected by a clinician, and can be drug-specific and/or therapy-specific. Profiles, segments of profiles, protocols and settings can be embodied in various forms, including but not limited to, code, equations, algorithms, and/or other expressions of machine readable code. A user such as a patient, pharmacist, scientist, drug program designer, medical engineer, nurse, physician, or other medical practitioner or healthcare provider can use infusion pump 100 to download delivery profiles from, for example, a computer management system or server on which a database or “library” of delivery profiles can be stored and accessed. In embodiments, a user can access and/or transfer delivery profiles or segments of profiles that accurately model natural, physical variances, such as profiles created using fourth degree polynomials, as shown in FIG. 3A (i.e., f(x)=ax⁴+bx³+cx²+dx+e). Additionally, subsystem software can allow a user to access a predefined delivery profile and divide the profile into segmentable delivery profiles, as shown in FIG. 3B. In embodiments, a user can integrate entire predefined delivery profiles, as shown in FIG. 3C, in addition to segments of profiles, as shown in FIG. 3B, to create a segmented delivery profile, depending, for example, on the device on which the delivery protocol will execute as well as the pharmacokinetics of the infusates being delivered by pumps 100. This integration of profiles, and/or segments of profiles, can be done by establishing rules for the profiles or segments to be run sequentially and/or in parallel by resolving the rules of the profiles or segments. The profiles or segments also can be integrated so as to cause them to be executed iteratively or in other ways. In addition, the profiles or segments can be integrated so as to cause them to deliver fluid according to some established relationship with the execution of other segments or other stimulus. These and other types of programmatic integration also can include iteration loops, profile segments that are specified or defined as modifications of a previous segment, and other inter-segment and/or intra-profile relationships. Additional examples of delivery methods that include various types of integration of profiles and/or segments are discussed in more detail herein below. In embodiments, the infusion pump delivery profile can also comprise a Programmed Intermittent Bolus (PIB) in which a user can directly initiate changes in infusion rates without altering polynomial parameters, such as polynomial coefficients.

In embodiments, each segment can be defined with a name, a set of delivery shape parameters, machine readable code, and/or user-defined parameters, which can be used as an underlying template for the execution of delivery protocols commonly supported by infusion pump devices (e.g., TCI, PCA, Total Parenteral Nutrition (TPN) tapers, Insulin on-board corrections, Intermittent Volume Over Time (IVOT), Boluses, etc.). Infusion pump profiles and segments of profiles (e.g., represented graphically in the examples of FIGS. 3A-C and 4A) can be embodied in various forms, including but not limited to, code, equations, algorithms, and/or other expressions of machine readable code, which can be transferred among electronic devices. In embodiments, a user can access and transfer to an infusion pump a predefined infusion profile that is typically executed on an infusion pump that is currently available commercially. A user can execute the predefined delivery protocol or protocols of a variety of different infusion pump devices, including, but not limited to one or more of weight-based protocols, intermittent delivery protocols, continuous delivery protocols, and delivery protocols with optional delivery features including, but not limited to, loading dose, bolus dose, replacement bolus, additive bolus, flush, volume limit, Keep Vein Open (KVO) rate, and TCI. In further embodiments, a user can reconfigure or edit a predefined delivery profile or segments of profiles by, for example, altering the polynomial coefficients underlying an infusion profile (e.g., altering a, b, c, d, and/or e in the fourth degree polynomial shown in FIG. 3A). In this way, a user (e.g., a researcher or clinician) can test various experimental infusion profiles and simulate various infusion work flows. For example, with reference to the fourth degree polynomial in FIG. 3A, when x is zero, and a, b, c, and d are zero, the delivery profile is flat (i.e., linear, as f(x)=e). Additionally, when a, b, and c are zero, the delivery profile models the process of ramping up between different infusion rates (i.e., f(x)=dx+e) to manage, for example, TPN volumes. In yet further embodiments, a user can interact with an infusion device (e.g., via a touchscreen, keypad, microphone, etc.) to alter the shape or slope of a delivery profile or segments of profiles or even adjust or modify a delivery profile. For example, modification may be desired after the initial “build” or definition of a profile, including even while a delivery profile is running on an infusion pump, for reasons including but not limited to PCA or a clinician-initiated bolus, TCI adjustments, rate adjustments, volume limit adjustments, quick start transitions, and clearing of programmed volume delivered (PVD). In some embodiments, a series of “if/then” statements, or questions and branching, can be used to enter various modifications, and these and other modifications can be made using an infusion pump itself, rather than having to return to a PC or server. In still other embodiments, a profile or underlying polynomial can have as an input, and/or be edited or altered based on, data received from other devices or systems. In some embodiments, this data can be feedback from sensors or other components or devices, as permitted within applicable safety rules, standards and limits. In a further embodiment, characteristics or information affecting linearity can be automatically considered. For example, patient weight can be a non-linear factor affecting rate and/or dosage of some drugs and therapies.

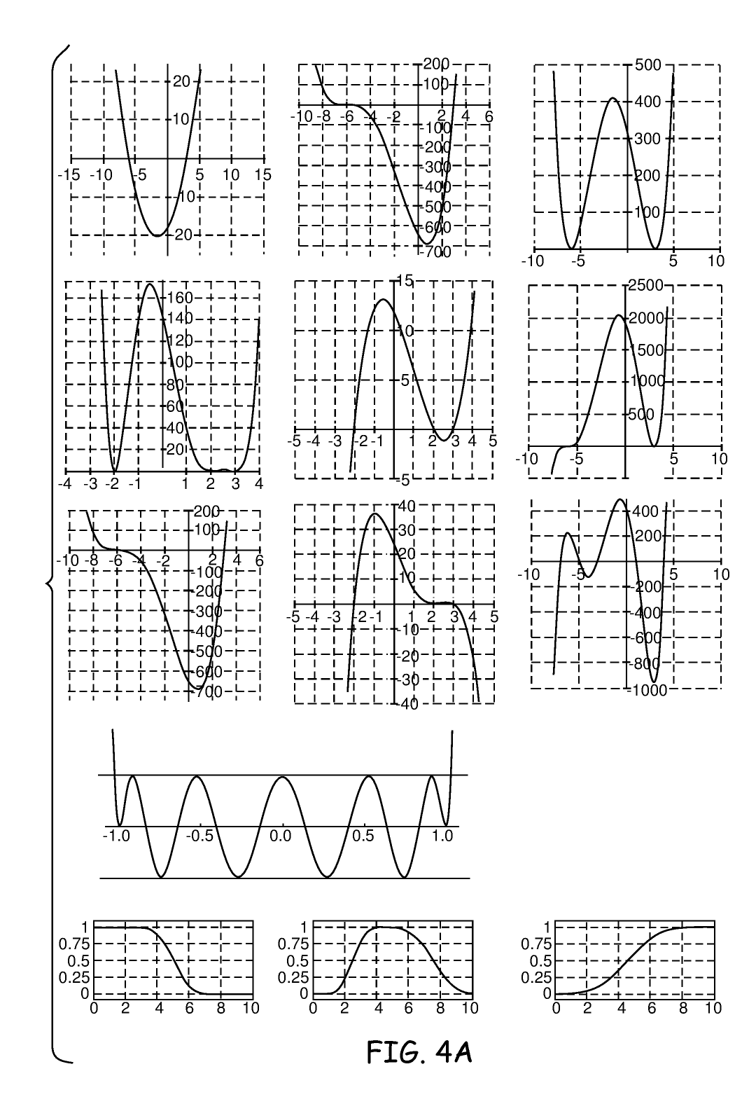

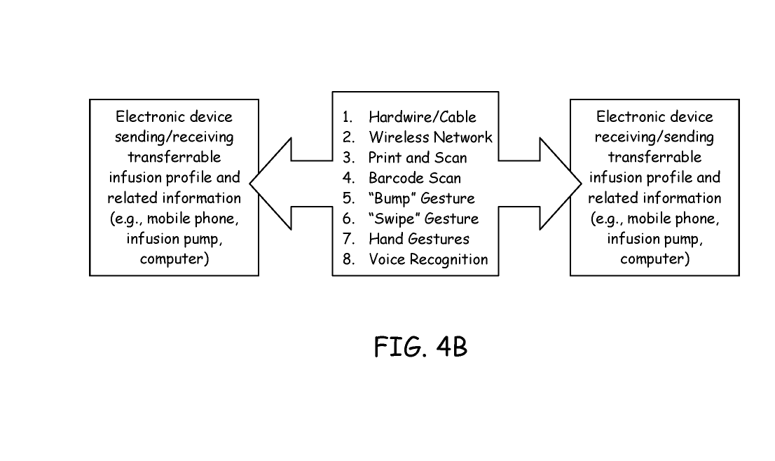

FIG. 4A represents a collection of infusion pump profiles. In embodiments, collections of infusion pump profiles or segments of profiles can be aggregated into a customized database or library of drug delivery profiles for use, for example, at a specific pharmacy or healthcare facility. The collections of infusion pump profiles or segments of profiles can be aggregated from the libraries or databases of various other infusion pump system manufacturers or healthcare providers in order to mimic or emulate the profiles used by those manufacturers or healthcare providers. In embodiments, the customized database or library of drug delivery profiles can also be created by various means and methods, including but not limited to, altering the polynomial coefficients underlying an infusion profile to create various shapes and configurations, as shown in FIG. 4A. The underlying polynomial equations can be first, second, third, fourth, fifth, sixth, seventh, eighth, ninth, or tenth degree polynomials—or polynomials of, potentially, any desired degree. In some examples, the underlying polynomial can be defined or altered to resolve to the instantaneous rate or to the cumulative amount delivered. This can be done through integration or other techniques discussed herein. The shapes or configurations of the drug delivery profiles can have negative or positive slopes. The shapes or configurations of the drug delivery profiles can also resemble a stepped up or stepped down configuration, or combinations thereof. The shapes or configurations of the drug delivery profiles can be regular or repeating, and/or irregular and stochastic. In embodiments, the customized database or library of drug delivery profiles can be created by overlaying different drug delivery profiles and then integrating them using an infusion pump or computer management system. In further embodiments, infusion pump profiles or segments of profiles can be created to mimic or emulate the infusion profiles and protocols of various other infusion pump system manufacturers or healthcare providers.

As shown in FIG. 4B, infusion pump profiles or segments of profiles can be created and transferred among various electronic devices, as well as among computer management systems and central servers. Infusion pump profiles and segments of profiles (e.g., represented graphically in the examples of FIGS. 3A-C and 4A) can be embodied in various forms, including but not limited to, code, equations, algorithms, and/or other expressions of machine readable code. Infusion profiles can be transferred as entire profiles, as segments, or as part of a larger report containing information relating to the infusion profile or segments of profiles, such as drug identity, pharmacokinetic characteristics, polynomial equations, curve shape or configurations, and execution or delivery protocols. Generally, such configurations, which can be downloaded from a computer and/or server and included in the configuration by PC- or software-based medication safety software, contain the definitions that enable an infusion pump or other medical device to provide the defined infusions. One example is the Toolbox/MSS PC (“Toolbox/Medication Safety Software—Personal Computer”) application, available from SMITHS MEDICAL ASD, though other software, hardware and applications also can be suitable. In embodiments, transferring (e.g., uploading and/or downloading) infusion pump profiles among electronic devices can be done, for example, by physically connecting one device to another using a cable or cables and initiating the transfer. In other embodiments, an infusion profile can be transferred wirelessly over a network (e.g., Bluetooth). In other embodiments, an infusion profile can be transferred by first printing out the infusion profile to be transferred and then scanning that profile into another electronic device. In further embodiments, the infusion profile to be transferred can be associated with a recognition system or an identifying feature. For example, the infusion profile to be transferred can be associated with a barcode, such that the barcode can be scanned by other electronic devices, resulting in the transfer of the infusion profile or segments of the profile, as well as other related information. In embodiments, databases or libraries of infusion profiles or segments of profiles can be created or aggregated such that each profile or segments of a profile can be associated with an identifying feature (e.g., a barcode). In further embodiments, infusion profiles can be transferred among electronic devices using a touch-based (e.g., keyboard or touchscreen) or a touchless (e.g., hand gestures or voice recognition) interface. In further embodiments, a user can interact with the user interface of an infusion pump to transfer an infusion profile by “swiping” with a forefinger or by “bumping” the pump with another electronic device such that the interaction facilitates the transfer of the infusion profile.

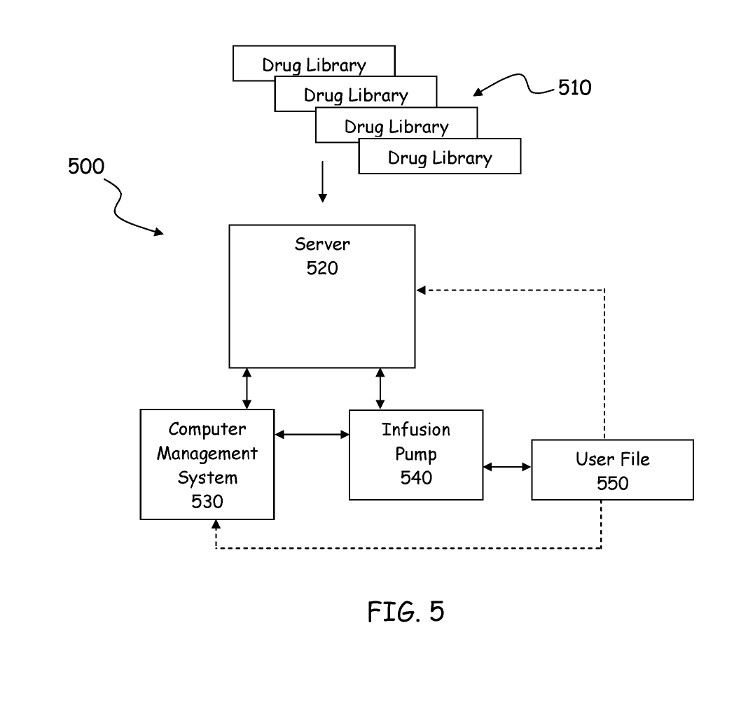

FIG. 5 is a flowchart of a network 500 showing interactions among various elements of an infusion pump system. In embodiments, infusion pump 540 can be programmed or configured with subsystem software to access databases 510 (often referred to as “drug libraries”) containing information, for example, relating to medications that can be used with a certain pump, as well as information corresponding to dosing guidelines, drug concentrations, dose limits, and clinical advisories. Additionally, databases or drug libraries 510 can contain drug delivery profiles that can be stored on server 520. Server 520 can be accessed by computer management system 530 (e.g., a hospital or pharmacy), which allows the users of computer management system 530 to transfer, edit, and/or integrate drug delivery profiles or segments of profiles.

In further embodiments, the user of infusion pump 540 can connect either to computer management system 530 or to server 520 to transfer, edit, and/or integrate drug delivery profiles or segments of profiles using infusion pump 540. A user can create a segmented delivery profile or a group of segmented delivery profiles that can be saved locally in user file 550 of infusion pump 540, or uploaded to server 520 or computer management system 530.

In embodiments, databases or drug libraries 510 can be stored as encrypted files or be part of an open source or other platform. A set of drugs within drug libraries 510 can, for example, be selected to be representative of the range of drugs used in healthcare facilities. The subsystem software can include an application program providing a user interface that enables an authorized individual (i.e., one with authorized password and access level) to transfer, edit, or integrate drug delivery profiles or segments of profiles specific to that user. Such user-generated drug delivery profiles can be stored as user files 550. Users of computer management system 530 can oversee user-generated delivery profiles as well as transfer, edit, and/or integrate drug delivery profiles or segments of profiles. Computer management system 530 can create or aggregate a customized library of drug delivery profiles for use, for example, at a specific pharmacy or healthcare facility. In embodiments, computer management system 530 can aggregate libraries of predefined delivery profiles executed on various infusion pumps from various different manufacturers. In further embodiments, computer management system 530 can create, edit, or integrate a library of delivery profiles corresponding to delivery shapes, profile segments, and polynomial expressions.

In embodiments, databases or drug libraries 510 comprising delivery profiles can enable users of computer management system 530 or infusion pump 540 to execute delivery protocols on infusion pump 540 without downloading new firmware or modifying the device executable. Generally, firmware (i.e., embedded software) contains the means to support pump programming and communication with a computer management system or central server. Typically, the operating system and communication protocols used to execute drug delivery profiles are stored on an infusion pump as nonvolatile Read Only Memory (ROM). In some embodiments, ROM can be replaced or supplemented by other components such as, e.g., Random Access Memory (RAM) and/or Flash memory. Many infusion pump systems currently available require new firmware upgrades or installation in order to execute new delivery protocols or protocols not typically executed on a particular device. In embodiments, databases or drug libraries 510 comprising delivery profiles can enable users of computer management system 530 or infusion pump 540 to execute new or non-native delivery protocols by mimicking or emulating existing infusion profiles used by various pump manufactures or healthcare providers, without a firmware upgrade or installation.

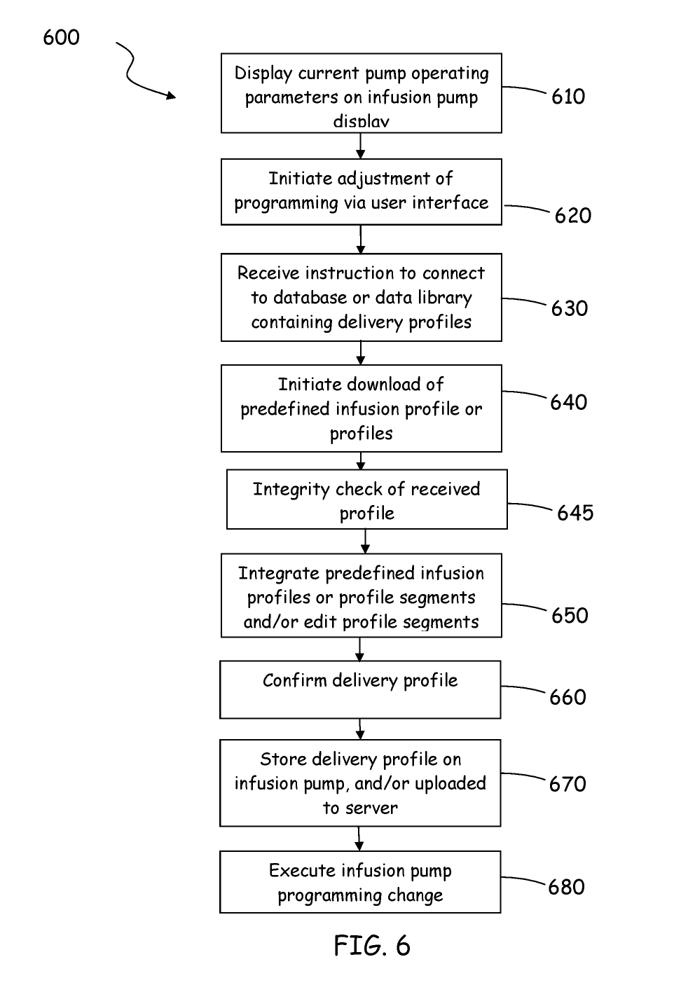

FIG. 6 provides a general flowchart of a method 600 for downloading, editing, and/or integrating drug delivery profiles or segments of profiles to an infusion pump. First, at 610, a user can visualize the current operating parameters displayed on an infusion pump. The display can be accessed via touchscreen technology, a keypad, or other means of interfacing with computer hardware. Next, at 620, a user can initiate the process of adjusting the current delivery protocol that will be executed on the infusion pump. This process of adjustment can involve editing and/or integrating delivery profiles or segments of profiles. Next, at 630, a user may receive an instruction to initiate the process of downloading from a server or computer management system a drug delivery profile, which can be a predefined profile or segments of predefined profiles typically used for executing delivery protocols on current medical devices. Next, at 640, a user can initiate downloading of the desired predefined delivery profiles or segments of profiles to an infusion pump. Next, at 645, an integrity check can be conducted on the received profile data. In an embodiment, the accuracy and reliability of the profiled received can be assessed and verified. In some embodiments, if the accuracy or reliability of the profile is below an acceptable threshold, the pump or computer system will prevent the profile from being run as part of an infusion protocol. Next, at 650, a user can edit or integrate entire profiles or segments of profiles to create a segmented delivery profile with the desired parameters. This can be done by interacting with a user interface (e.g., a touchscreen or keypad), and for example, altering the coefficients of the underlying polynomial expression of the profile or by changing the slope or shape of the curve. Next, at 660, a user can confirm the delivery profile so created. Next, at 670, the delivery profile can be saved and stored in a user file on the infusion pump and/or it can be uploaded to a computer management system or central server (see, e.g., FIG. 5).

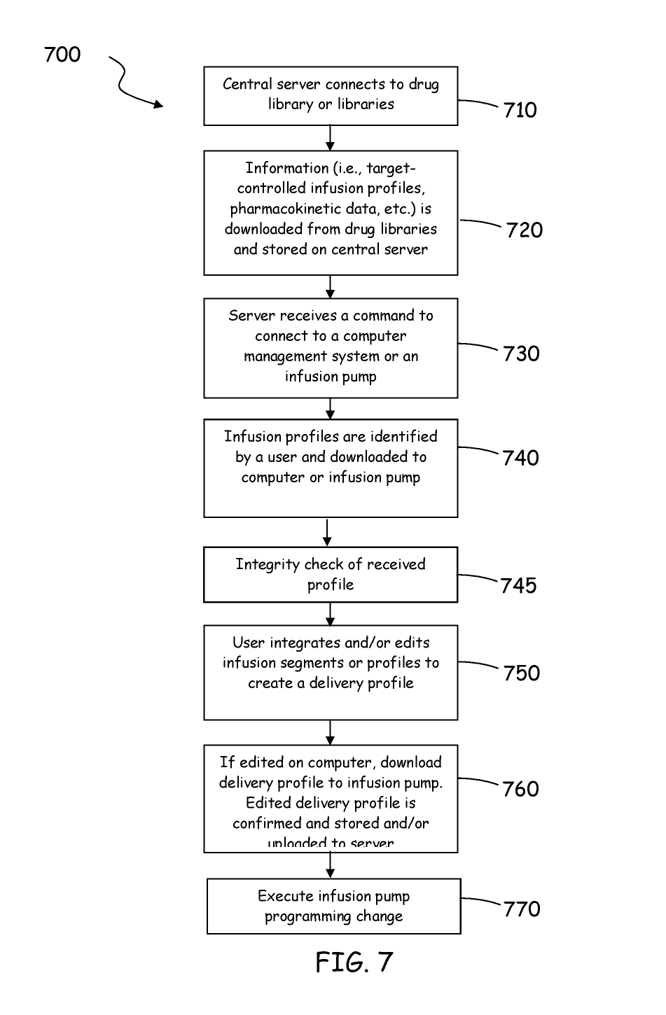

At 680, once the safety and efficacy of the delivery profile is properly verified by a healthcare provider, the delivery profile can be executed as part of a delivery protocol. FIG. 7 provides a general flowchart 700 for downloading, editing, and/or integrating drug delivery profiles or segments of profiles from a server for execution on an infusion pump. First, at 710, a central server connects to a database or library containing drug delivery profiles. Next, at 720, parameters relating to the drug delivery profiles in the library (e.g., infusion rates, concentrations, pharmacokinetic data, etc.) can be downloaded to a central server and stored therein. Next, at 730, the server containing the drug delivery libraries receives a command from a user to connect to a computer management system or an infusion pump (see, e.g., FIG. 5). Next, at 740, the user identifies the relevant delivery profile and downloads the profile to a computer or infusion pump. In embodiments, the downloaded profile can be divided into segments prior to downloading, or the downloaded profile can be divided into segments by the user on an infusion pump. Next, at 745, an integrity check can be conducted on the received profile data. In an embodiment, the accuracy and reliability of the profiled received can be assessed and verified. In some embodiments, if the accuracy or reliability of the profile is below an acceptable threshold, the pump or computer system will prevent the profile from being run as part of an infusion protocol. Next, at 750, the user can integrate different delivery profiles or segments of profiles to create a delivery profile. This can be done by a user of a computer management system or the user of an infusion pump. Additionally, the user can edit the delivery profile by altering the coefficients of the underlying polynomial expression or by changing the slope or shape of the curve by interacting with a user interface (e.g., a touchscreen). Next, at 760, if the delivery profile was edited on a computer or remote device, the delivery profile can be downloaded to the infusion pump. In embodiments, the edited delivery profile is confirmed and stored and/or uploaded to a computer management system or central server (See, e.g., FIG. 5). At 770, once the safety and efficacy of the delivery profile is properly verified by a healthcare provider, the delivery profile can be executed as part of a delivery protocol. The infusion pump executing the profile can be further configured to calculate the desired infusion rate and update blood and target site concentrations as the infusion pump operates. Additionally, the infusion pump executing the profile can be further configured to allow one infusion to send the current or most recently used rate to the next infusion for flow continuity.

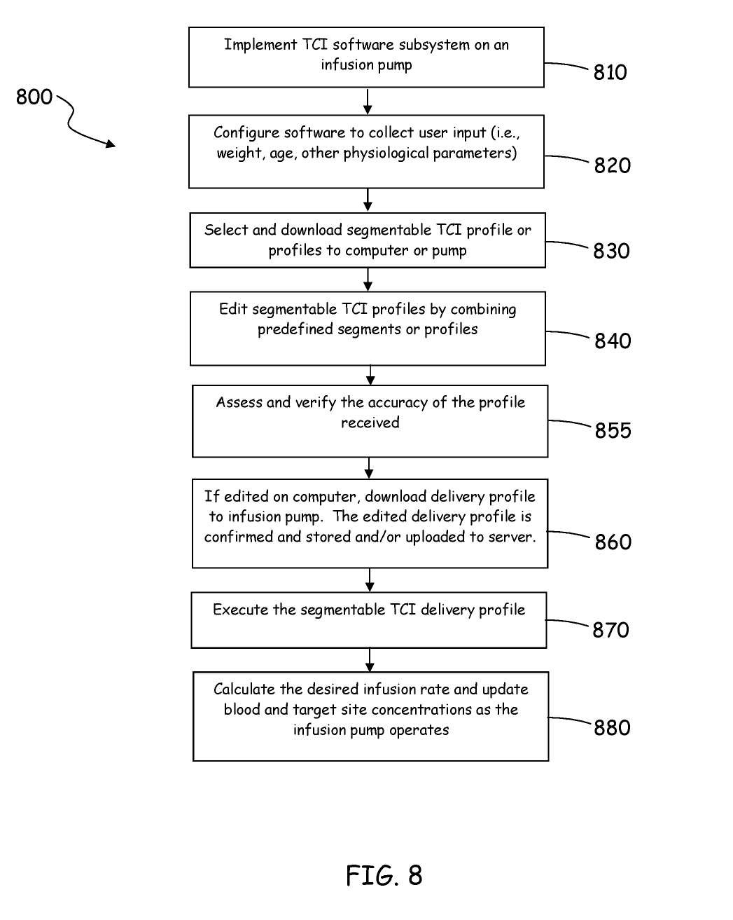

In embodiments, delivery profiles can be executed as part of TCI systems. FIG. 8 provides a general flowchart of the steps 800 for downloading, editing, and/or integrating delivery profiles or segments of profiles for executing with a TCI system. First, at 810, a TCI software subsystem is implemented on an infusion pump. Next, at 820, the software is configured to collect user input (e.g., physiological parameters like age, weight, etc.), which can be entered by the user or other healthcare professional by interacting with the user interface of a computer management system or an infusion pump (see, e.g., FIG. 5). Next, at 830, a user can select at least one TCI profile or segments of at least one profile and download it or them to a computer management system or directly to an infusion pump. Next, at 840, the user can edit TCI profiles or segments of profiles by combining or integrating one or more predefined profiles or segments of profiles to create a delivery profile. Optionally, in embodiments, the user can edit a delivery profile by altering polynomial coefficients or by altering segment shapes and slopes, as described previously. Next, at 855, the accuracy and reliability of the profiled received can be assessed and verified. In some cases, if the accuracy or reliability of the profile is below an acceptable threshold, the pump or computer system will prevent the profile from being run as part of an infusion protocol. Next, at 860, if the delivery profile was edited on a computer or remote device, the delivery profile can be downloaded to the infusion pump. In embodiments, the edited delivery profile is confirmed and stored and/or uploaded to a computer management system or central server (see, e.g., FIG. 5). Next, at 870, the delivery profile can be executed as part of a TCI delivery protocol, once the safety and efficacy of the delivery profile is properly verified by, e.g., a healthcare provider and/or by the infusion pump system itself. At 880, the infusion pump executing the TCI profile can be further configured to calculate the desired infusion rate and update blood and target site concentrations as the infusion pump operates. Additionally, the infusion pump executing the TCI profile can be further configured to allow one infusion to send the current or most recently used rate to the next infusion for flow continuity.

Various embodiments provide increased flexibility and options for intra-system and inter-system operability and functionality. As previously mentioned, profiles or underlying polynomials can have as an input, and/or be edited or altered based on, data received from other devices or systems, such as feedback from sensors or other components or devices, as permitted within applicable safety standards and limits. As such, embodiments can be used as a building block for a closed-loop or feedback-based system, including one enabling a responsive or reactive profile based on various conditions or information as they may occur in real time, are sensed, recorded, entered or otherwise obtained, and are processed and incorporated by and into the underlying polynomial and/or profile. In some embodiments, clinician approval, monitoring or other involvement can be required before any delivery changes are implemented. In still other embodiments, predictive elements can be incorporated, such as by using sensors to obtain feedback and predict future needs or events based on past feedback, performance or real-time current information. Still other predictive embodiments can use past treatment data, such as data related to patient treatment or response, to provide future therapies.

Various embodiments also can be used within or as part of various delivery methods, which can be defined sets of delivery sequences and associated rules for programming and running a method, and can include optional workflow elements. Examples of delivery methods include continuous and intermittent delivery methods. An example continuous delivery method can include several segments, including a loading segment, a main segment, optional replacement clinical bolus segment(s), and optional KVO segment(s), and can include different infusion types with different units for programming dosages (e.g., mL/hr; dose/kg/min; etc.). An example intermittent delivery method can include a main segment and optional flush segment(s), and also can include different infusion types with different units for programming dosages (e.g., mL/hr; dose/kg/min; etc.). Conventional continuous and intermittent delivery methods are hard coded to run particular combinations of segments and for each segment type.

In contrast, features and advantages of and provided by embodiments of the devices, systems, methods and techniques discussed herein relate to enabling user definition of delivery methods, profiles and segments. In embodiments, rules for a segment can be defined from a set of operating rules; the names of segments can be defined from a name rule set; inter-segment behaviors can be defined from an operating rule set; and infusion types that are available for a plan can be defined from a set of types. Advantageously, any number of sequential or parallel segments can be defined, providing a user with increased flexibility and ability to tailor a profile for a patient, setting, use, or according to some other factor or combination of factors.

Furthermore, embodiments can implement or be compatible with a variety of delivery methods, including the continuous and intermittent methods discussed above, but also others. For example, as previously mentioned some embodiments can be used in various delivery methods that integrate or resolve profiles or segments, such as those that involve multiple delivery rates sequentially or in parallel, in various different ways. Embodiments that include integration of profiles and/or segments can link the profiles or segments in logical ways and reduce the need for manual resolution, such as by clinicians or through hard programming. Several examples that include various types of integration follow.

In a “least” delivery method of integrating multiple delivery rates, the delivery at each instant is performed at the least of the set of rates. For example, if a first delivery rate is a constant rate of 1 mL/hr and a second delivery rate is a linearly increasing rate starting at 0 and ending 10 minutes later at 5 mL/hr, integrating at “least” would mean delivering at a linearly increasing rate starting at 0 and reaching 1 mL/hr after 2 minutes. Once the rate of 1 mL/hr is reached, delivery continues at that rate. An example use of this would be delivery of a substance that requires gradual increase or decrease in delivery but must never exceed a delivery rate to avoid overdosing.

In a “greatest” delivery method of integrating multiple delivery rates, the delivery at each instant is performed at the greatest of the set of rates. For example, if delivery is a constant rate of 1 mL/hr and another is a linearly increasing rate starting at 0 and ending 10 minutes later at 5 mL/hr, integrating at “greatest” would mean delivering at rate of 1 mL/hr until the linearly increasing rate passes 1 mL/hr at the 2 minute point and then linearly increasing for the remainder of the 10 minutes. An example use of this would be delivery of a substance that requires gradual increase or decrease in delivery but must never go below a minimum to avoid loss of the fluid path in the body.

Other delivery method examples include serial concatenation, including serial concatenation with smoothing or splining. Refer, for example, to FIGS. 3A-3C. Smoothing and/or splining can include algorithmic or mathematic adjustments of the delivery rates near the edges of adjacent segments to facilitate or ease transitions therebetween. Additionally, some of the examples used herein are additive or subtractive, including additive methods that involve one or more negative numbers. Refer, for example, to FIGS. 3 and 4, which depict profiles and segments that include negative slopes. Still other delivery method examples can include absolute, in which delivery is carried out at the absolute value of the delivery rate of all of the profiles or segments being executed simultaneously; programmatic, which can include iteration loops, “if/then” statements, profile segments that are specified or defined as modifications of a previous segment, and other inter-segment and/or intra-profile relationships; and reactive, which can include the shape of a delivery profile being altered or affected by changes in another profile or an external stimulus. Both programmatic and reactive delivery methods were also mentioned above.

Another feature and advantage of embodiments is the ability to use the devices, systems, methods and techniques discussed herein in virtual ways, such as with a virtual pump or infusion system. A virtual pump can comprise a set of software operating on a server or computer that enables a user to test or evaluate delivery methods, profiles, segments and other features. This can be advantageous in clinical learning and educational settings, and for research purposes related to patient care, drugs, hardware and other factors. For example, researchers or clinicians can use the virtual pump to create delivery profiles and then run them through a virtual pump or multiple virtual pumps to see how the delivery profile performs on its own or how multiple delivery profiles interact. Researchers and clinicians also could use a virtual pump for simulations, testing, drug development, education of medical professionals, and for other purposes. In embodiments, the virtual pump can be configured to operate on a rules basis, accept lower or higher parameters, permit ambiguity, and otherwise run using incomplete or conflicting information, in order for a research or clinician to evaluate characteristics, performance and other factors in a highly sophisticated manner that does not affect patients, equipment or controlled substances. A variety of simulations can be run, and in embodiments the virtual pump can document the various settings and results for ease of evaluation. In still other embodiments, the data and settings underlying profiles or methods evaluated on the virtual pump and approved for implementation in actual clinical settings can be exported from the virtual pump to a server or computer where they can be made available for use on infusion pumps. Security features and settings can be implemented in the virtual pump to ensure that only authorized data is made available more broadly than on the virtual pump. In further embodiments, the virtual pump is isolated so as to not enable sharing or comingling of data with that of live pumps.

It is to be appreciated and understood that methods, systems, and software for downloading, editing, and/or integrating drug delivery profiles or segments of profiles, such as have been described by example or otherwise contemplated herein, may allow for delivery of arbitrarily complex patterns, as the profiles and their subsequent deliveries are conducted in bursts or stages. Therefore, delivery based on what will be due by an arbitrary point in time makes complex profiles easier to deliver.

It is further to be appreciated and understood that any of the aforementioned delivery profiles or segments of delivery profiles can be stored and/or performed in the infusion pump itself or a computer server, in the pump internally or separately or otherwise remotely from the pump. Further, it is to be appreciated that the aforementioned delivery profiles or segments of delivery profiles can be created by or with outside software or systems and subsequently downloaded to or integrated with the systems and software described herein.

For example, in an embodiment a database comprises at least one delivery profile executable as part of a medical device delivery protocol, wherein the at least one delivery profile comprises at least one profile segment integrated to form the at least one delivery profile. The at least one delivery profile executable as part of a medical device delivery protocol can be used to control, or cause to operate, a medical device, such as an infusion pump. In embodiments, providing at least one delivery profile to a medical device, such as an infusion pump, can configure or reconfigure the medical device to provide a therapy to a patient, such as through infusion of a fluid, drug, infusate or other medical material deliverable by the medical device according to the at least one delivery profile. The at least one delivery profile and/or the at least one profile segment can be created or programmed at the device, or can be communicated, partially or wholly, to the pump from a processor, computer, server, medical device, handheld device, and/or other external device via a communications network or device, and wired, wirelessly or a combination thereof. Similarly, a delivery profile executable on an electronic device as part of a medical device delivery protocol, the delivery profile comprising at least one profile segment integrated to form the delivery profile, can configure or reconfigure the medical device and/or cause the medical device to operate to provide a therapy or treatment to a patient by delivering a medical fluid or other substance to a patient according to the delivery profile.

In embodiments, an infusion pump comprising programmable circuitry configured to download at least one delivery profile or at least one profile segment; integrate the at least one delivery profile or the at least one profile segment to form an executable delivery profile; and execute a medical device delivery protocol comprising the executable delivery profile on the infusion pump, can operate to deliver a therapy or treatment to a patient when executing the medical device delivery protocol. The database and infusion pump mentioned above can operate in embodiments as part of a medical device system that also configures or reconfigures the infusion pump and cause the infusion pump to operate and deliver a therapy or treatment to a patient.

In embodiments, a method of creating a varied segmentable delivery profile can comprise accessing a database comprising at least one segmentable delivery profile or at least one profile segment; transferring the at least one segmentable delivery profile or the at least one profile segment to an electronic device; and integrating the at least one segmentable delivery profile or the at least one profile segment to create a second delivery profile. This and other methods also can include executing the second delivery profile as part of a delivery protocol of the infusion pump to cause the infusion pump to operate and provide a therapy or treatment to a patient by delivering a fluid, drug, infusate or other material to the patient according to the delivery protocol.

These examples are given according to only some of many possible embodiments, keeping in mind that some embodiments relate to virtual devices or machines implemented using computers, processors, medical devices, or other devices that enable a user to cause a medical device to operate virtually via one of these other devices or machines for the purposes of simulating or testing operation of the medical device.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the subject matter hereof as set forth in the appended claims and the legal equivalents thereof. For example, in embodiments described with a syringe-type infusion pump, it is to be understood that an ambulatory type pump could have been alternatively employed.

The embodiments above are intended to be illustrative and not limiting. Additional embodiments are within the claims. Although subject matter hereof has been described with reference to particular embodiments, workers skilled in the art will recognize that changes may be made in form and detail without departing from the spirit and scope of the subject matter.

Various modifications to subject matter hereof may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the subject matter. Likewise, the various features described above should all be regarded as example embodiments, rather than limitations to the scope or spirit of the subject matter. Therefore, the above is not contemplated to limit the scope of the subject matter.

For purposes of interpreting the claims for subject matter hereof, it is expressly intended that the provisions of Section 112, sixth paragraph of 35 U.S.C. are not to be invoked unless the specific terms “means for” or “step for” are recited in a claim.

1-28. (canceled)

29. A medical device system, comprising:
a database comprising at least one delivery profile or at least one profile segment, wherein the at least one delivery profile or the at least one profile segment are integrated to form an executable delivery profile;
an infusion pump configured to execute a medical device delivery protocol comprising the executable delivery profile;
a computer management system; and
a network operably coupled to the database, the infusion pump, and the computer management system.

30. The medical device system of claim 29, wherein the at least one delivery profile or the at least one profile segment are transferred via the network to at least one of a second infusion pump, a computer, a server, or a mobile device.

31. The medical device system of claim 29, wherein the at least one delivery profile and the at least one profile segment are stored on a server as part of an open source database of delivery profiles and profile segments.

32. The medical device system of claim 29, wherein the at least one delivery profile and the at least one profile segment are predefined as part of existing medical device delivery protocols.

33. The medical device system of claim 29, wherein the at least one delivery profile and the at least one profile segment mimic existing medical device delivery profiles.

34. The medical device system of claim 29, wherein the delivery protocol is a TCI delivery protocol.

35. The medical device system of claim 29, wherein the at least one delivery profile is executed on the infusion pump without changing the firmware.

36. The infusion pump of claim 29, wherein the at least one delivery profile or the at least one profile segment are edited by interacting with a user interface of at least one of the infusion pump, a computer, or a mobile device.

37-44. (canceled)

45. A method of operating an infusion pump comprising:
accessing a server or computer management system;
transferring at least one delivery profile or at least one profile segment of the at least one delivery profile to the infusion pump;
integrating the at least one delivery profile or the at least one profile segment to create a second delivery profile; and
executing the second delivery profile as part of a delivery protocol of the infusion pump.

46. The method of claim 45, wherein the at least one delivery profile and the at least one profile segment are either predefined as part of existing medical device delivery protocols, or mimic existing medical device delivery profiles.

47. (canceled)

48. The method of claim 45, wherein the at least one delivery profile and the at least one profile segment are stored on a server as part of an open source database of delivery profiles and profile segments.

49. The method of claim 45, wherein the at least one delivery profile is further transferred to an electronic device.

50. The method of claim 45, wherein the at least one delivery profile is stored on the infusion pump or uploaded to a server or computer management system.

51. The method of claim 45, further comprising editing the at least one delivery profile or the at least one profile segment by interacting with a user interface of at least one of an infusion pump, a computer, or a mobile device.

52. The method of claim 45, wherein the execution of the at least one delivery profile is part of a TCI protocol.
